(19) United States
(12) Patent Application Publication
(10) Pub. No.: US 2002/0132030 A1
(43) Pub. Date: Sep. 19, 2002

(54) Composition and method for prolonging the useful life of enteral feeding tubes

(76) Inventors: Lester David Michels, Eden Prairie, MN (US); David Curtis Egberg, Shorewood, MN (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/092,637

(22) Filed: Mar. 7, 2002

Related U.S. Application Data
(63) Continuation of application No. 09/345,123, filed on Jul. 6, 1999.

(51) Int. Cl. C12H 1/10

(52) U.S. Cl. 426/321

(57) ABSTRACT

An ingestible composition to prolong the useful life of an enteral feeding tube and a system of an enteral feeding tube and the composition is provided. The complete enteral feeding solution contains an edible antimicrobial agent which is 0.05% to 1.0% by weight alkyl paraben, the alkyl group having 1-10 carbon atoms. Methyl, ethyl, or propyl paraben are particularly preferred.

This invention relates to methods and compositions for prolonging the useful life of enteral feeding tubes.

More particularly, the invention relates to a method and composition for prolonging the useful life of an enteral feeding tube while the tube remains inserted in a patient.

In a further respect, the invention relates to an enteral feeding composition which can be safely ingested by a patient, but which inhibits microbial growth.

### BACKGROUND

The administration through enteral feeding tubes of aqueous solutions of nutritionally balanced food compositions is known in the art.

Enteral feeding formulas and devices can provide favorable environments for contamination and support of growth for microorganisms, especially when the materials are not properly handled or managed. The formulas provide medium and nutrients necessary to support growth, and the devices provide ambient conditions (e.g., surfaces, warm temperatures, etc.) which can accelerate the colonization of the microorganisms. As a consequence, any errant contamination of the enteral feeding system can lead to a significant rise in contaminants unless proper care and precautionary measures are taken; even so, contamination of enteral feeding systems continues to be problematic in health care facilities.

During the use of enteral feeding tubes, residual food proteins, starches, and other components often accumulate in and may block the feeding tube. Such accumulation and blockages provide a ready site for the growth and multiplication of microorganisms. Cleaning of feeding tubes may be performed by flushing with water and in some cases enzymes (e.g., meat tenderizer) or other agents that may dissolve residual food blockages or accumulations. However, cleaning is usually incomplete and tubes eventually may become partially or completely occluded, or the tube material may deteriorate and fail.

Accordingly, it would be highly desirable to provide a method for reducing the incidence of, or preventing blockages and material degradation in an enteral feeding tube that would maintain and prolong the useful life of the feeding tube and avoid tube replacement.

Many different types of microorganisms have been identified in enteral feeding systems and have been found in the formula container, the delivery set, and the patient's feeding tube (and stomach). Among those identified are yeasts and fungi such as Aspergillus niger and Candida albicans. Candida has commonly been found to be present in contaminated feeding systems and has been associated with severe degradation of feeding tubes. The source of the contamination is likely either the patient (stomach) or the caregiver (hands). Feeding tubes may deteriorate to the point of clogging due to microorganism overgrowth, material breakdown and discoloration. In some cases, patient (nosocomial) infection has been attributed to contaminated feeding systems.

Risks associated with microorganism contamination include overgrowth and clogging of patient feeding tubes, deterioration of feeding tube materials, curdling of formula in feeding containers/systems, and possible patient local site infection and source for nosocomial infections. Reducing or inhibiting the proliferation of microorganisms in the enteral-feeding formulas by use of various preservative methods has been applied in some commercial mixtures such as Vivonex™ (Novartis) and AMTF™ (Nyer). These methods have employed the fact that low pH (<6) and the presence of preservatives that function optimally at lower pH inhibits microbial growth. However, this approach is not always practical in many formulas where maintaining pH>6 is necessary to avoid protein denaturalization and formula curdling.

There are a number of food additives that display antimicrobial properties and are effective in inhibiting the growth of certain microorganisms. These additives include alkyl parabens, alkyl having 1-10 carbon atoms, including methyl and propyl paraben; also useful are benzoic acid and its derivatives, sorbic acid and its derivatives, propionic acid and its derivatives, lactic acid and its derivatives, alkyl carbonates, bisulfites, nisin, natamycin, alkyl sulfates and others. Some of these additives have been used individually and in combination to inhibit microbial growth in liquid and solid foods, but they have not been used in feeding tube compositions to inhibit the growth of microorganisms and to prevent clogging of the tube.

Therefore, it is a principal object of the invention to provide an improved method and composition for prolonging the useful life of an enteral feeding tube.

Another object of the invention is to provide a composition, which inhibits microbial growth in a feeding tube formula.

A still further object of the invention is to provide an enteral feeding tube system comprising a feeding tube and an enteral feeding tube microbial growth inhibition formula, which has prolonged life, does not utilize toxic cleaning agents and which can safely be ingested by a patient.

These and other further and more specific objects and advantages of the invention will be apparent to those skilled in the art from the following detailed description.

### SUMMARY OF THE INVENTION

To minimize or reduce fungal, yeast and other microorganism proliferation in patient feeding tubes and thereby extend the useful life of the enteral feeding system, this invention teaches the addition of the antimicrobial parabens (preferably lower alkyl having 1-10, or 1-5 carbon atoms; especially the methyl, ethyl or propyl, or heptyl parabens) to enteral formulas and delivery of the antimicrobial mixture to the lumen of a patient's feeding tube (as during normal continuous feeding) on a continuous or intermittent basis for this purpose

Furthermore, this invention demonstrates that lower alkyl, especially methyl and propyl parabens in effective inhibitory concentrations up to 1.0% will inhibit the growth of fungus, yeast and other microorganisms such as lactobacillus) and prolong the time that feeding tubes will remain patent when exposed to contamination in the formula. The use of the invention system of antimicrobial enteral formula together with a feeding tube, is also useful in reducing the deterioration of feeding tube materials, per se, by reducing and/or limiting the influence that microorganism overgrowth may have on the degradation process.

The precise ingredients of the enteral diet composition aspect of the present invention are not critical to the practice of the invention. Any suitable and commercial available diet can be employed. A complete feeding tube diet typically includes vitamins, minerals, trace elements as well as nitrogen, carbohydrate and fatty acid sources in a liquid form so that it can be used as the sole source of nutrition supplying essentially all the required daily amounts of vitamins, minerals, carbohydrates, fatty acids and the like. Any diet composition, whether as nutritional supplement or a total feeding, provides an energy supply of from about 10 to 3500 kcal/day, preferably 100-2000 kcal/day, and more preferably from about 150 to about 2000 kcal/day; and still more preferably 250-1500 kcal/day.

The following is a Table showing the Adult Standard Enteral Formula Composition:

TABLE 1
Adult Standard Enteral Formula Composition

| Contents | Typical range (per liter) |
|---|---|
| Protein (g) | 37-90 |
| Carbohydrate (g) | 130-220 |
| Fat (g) | 25-88 |
| Fiber (g) | 0-12 |
| Water (g) | 700-850 |
| Sodium (mg) | 620-1420 |
| Potassium (mg) | 1060-2100 |
| Vitamin A (IU) | 2800-11000 |
| Vitamin C (mg) | 51-400 |
| Thiamin (mg) | 1.3-3.2 |
| Riboflavin (mg) | 1.5-3.6 |
| Niacin (mg) | 11-43 |
| Calcium (mg) | 560-1268 |
| Iron (mg) | 9.5-19 |
| Vitamin D (IU) | 210-430 |
| Vitamin E (IU) | 17-120 |
| Vitamin B6 (mg) | 1.5-4.3 |
| Folic Acid (mcg) | 210-640 |
| Vitamin B12 (mcg) | 3-13 |
| Phosphorus (mg) | 530-1268 |
| Iodine (mcg) | 79-161 |
| Magnesium (mg) | 210-430 |
| Zinc (mg) | 8.5-32 |
| Copper (mg) | 1.1-2.11 |
| Biotin (mcg) | 160-480 |
| Pantothenic Acid (mg) | 5.3-21 |
| Vitamin K (mcg) | 38-110 |
| Choline (mg) | 56-600 |
| Chloride (mg) | 900-1600 |
| Manganese (mg) | 1.7-5.4 |
| Selenium (mcg) | 57-120 |
| Chromium (mcg) | 86-160 |
| Molybdenum (mcg) | 130-270 |
| Carnitine (mg) | 0-170 |
| Taurine (mg) | 10-280 |
| M-inositol (mg) | 10-1300 |
| Amino Acid (grams/ | |
| 100 grams protein) | Typical Range |
| L-Histidine | 1.9-2.9 |
| L-Isoleucine | 4.6-5.2 |
| L-Leucine | 8.1-10.9 |
| L-Lysine | 6.2-8.0 |
| L-Methionine | 1.3-2.8 |
| L-Phenylalanine | 3.8-5.3 |
| L-Threonine | 3.8-7.0 |
| L-Tryptophan | 1.0-1.7 |
| L-Valine | 4.6-6.7 |
| L-Alanine | 3.0-4.9 |
| L-Arginine | 2.8-5.5 |
| L-Aspartic Acid | 9.5-11.7 |
| L-Cystine/Cysteine | 0.4-2.5 |
| L-Glutamine* | 6.9-11.2 |
| L-Glutamic Acid | 8.9-12.5 |
| Glycine | 1.9-4.2 |
| L-Proline | 5.4-12.8 |
| L-Serine | 5.0-6.0 |
| L-Tyrosine | 2.9-5.5 |

*Based on theoretical calculation

Within the parameters of Table 1, there are many suitable complete enteral feeding formulas useful in this invention.

With reference to the components listed in Table 1, suitable nitrogen sources include proteins such as caseinates or protein hydroslyates or vegetable protein such as soy and amino acids, or combinations thereof. Suitable carbohydrate sources include various starches and maltodextrins. Carbohydrates can be selected from among the group consisting of digestible carbohydrates such as dextrose, fructose, sucrose, maltose, corn syrup solids, trisaccharides, tetrasaccharides, pentasaccharides, hexasaccharides, oligosaccharides and high saccharides, or mixtures thereof. Suitable fat sources include the triglycerides.

The standard enteral formula composition of Table 1 contains water, which makes the composition liquid enough to be used in the feeding tube situation. These compositions can also be supplied in the powered form that may be reconstituted with the water to make up the liquid. The powder is ordinarily partially dissolved and partially suspended in the resulting liquid form of the invention. While it is possible to reconstitute the composition with liquid such as alcohol, the reconstituting liquid will ordinarily be water. The water may contain additional ingredients such as alcohol, glycerol, propylene glycol, sugars and flavor.

Edible acidulants can also be utilized in the powder composition of the invention, and include malic acid, acetic acid, citric acid, lactic acid, fumaric acid, ascorbic acid, or an acidic salt such as sodium acetate in order to adjust the pH within the range of 2 to 6.9. An acid pH is useful because it reduces microbial activity and increases the effectiveness of some antimicrobial agents.

In a preferred embodiment of this invention, however, the pH can be from 3-8, and preferably pH>5.5, in order to avoid protein denaturalization and formula curdling.

When the powder rejuvenation composition of the invention is reconstituted with water, the amount of water used with a selected weight of powder can vary as desired. When, for example, it is desired to make the resulting aqueous solution more viscous so it better adheres to the internal surfaces of the feeding tube and to residual food in the feeding tube, the quantity of water can be reduced or the amount of fiber increased.

When it is desired to increase the viscosity of the solution that results after the powder composition is reconstituted with water, xanthan gum, carrageenan or another thickener can be included in the powder composition. Such thickeners are normally present in the powder rejuvenation composition in a concentration in the range of 0.50% to 12.0% by weight.

The preferred viscosity of the reconstituted enteral feeding tube composition will, as appreciated by those of skill in the art, vary depending on a number of factors. Such factors can include the size of the enteral tube, the food composition being administered through the tube, whether the tube is completely blocked, whether there is a large amount or a small amount of residual food, whether the reconstituted rejuvenation composition is administered with a food composition, etc. Even at low viscosities of 50 centipoises or less, the composition retains its homogeneity; the preferred viscosity is usually less than 50 centipoises.

Enteral tubes are used to dispense food into the esophagus, stomach, or intestinal tract of a patient.

The alkyl parabens, when alkyl is defined as having an 1-10 carbon atoms, preferably 1-5 carbon atoms, and most preferably methyl, ethyl, or propyl, can be present in the enteral feeding composition at from about 0.1% to about 1.0%, by weight based on the total weight of the feeding composition in its final liquid (or reconstituted) form.

Other antimicrobial agents can be employed in this invention, including benzoic acid salts and esters; sorbic acid salts and esters; propionic acid salts and esters; lactic acid salts and esters; alkyl carbonate (alkyl having 1-5 carbon atoms, such as dimethyl carbonate; bisulfite salts; nisin; natamycin and pimaricin; saponins; and diacetyl. The use of the term “salts” is defined as being the pharmaceutically acceptable salts, such as sodium, potassium, or the like. These will be useful at about the same levels as that of the alkyl parabens.

The following Examples illustrate the invention.

### EXAMPLE 1

### Paraben challenge in commercially sterile product.

Commercially sterile product (Isosource®) with 0.1% methyl or propyl paraben is prepared. Organisms were challenged in both variations at their optimum growth temperature and room temperature (RT). The populations are determined at different time intervals and compared to a spiked control without the parabens.

At 35° C., Staphylococcus aureus shows slightly lower levels than the spiked control (no paraben) at 24 hours but by 48 hours no inhibition is seen. At ROOM TEMPERATURE, both parabens yield lower levels than the control at 35C.

Inhibition of Enterobacter cloacae at 24 hours looks dramatic with methyl paraben at 35° C., but 0.5 logs difference in population is seen at 48 hours compared to the control. At room temperature, methyl paraben shows slight inhibition at 24 hours and increases by 48 hours with growth 1.5 logs lower than the propyl paraben sample.

At 24 hours, Lactobaccillus leichmanii levels are close to original inoculum levels. At 48 hours, both parabens are inhibitory at room temperature and 35° C. at levels 3-5 log cycles below the spiked control without paraben.

Aspergillus niger is inoculated at 530 colony forming units (CFU)/ml. This population could not be recovered from the samples containing paraben at all. A few CFU's were seen at 7 days but the only significant growth occurred at 14 days in the spiked sample without paraben.

Candida albicans is inhibited by propyl paraben at 7 days showing a 2-3 log cycle lower CFU/ml than the control, but by 14 days, this difference narrows to <2 logs.

### EXAMPLE 2

### A Challenge Study Using Combinations

A challenge study is done on commercially sterile product containing combinations of 4 GRAS antimicrobial agents: methyl paraben, propyl paraben, diacetyl, and natamycin. Ten different matrices are tested against 6 different organisms: A. niger, B. stearotherm, C. albicans, E. cloacae, L. Leichmanii, and S. aureus. The most effective inhibitory agent overall is 0.2% propyl paraben. It shows good inhibition of three out of the six organisms studied, and slight inhibition against S. aureus, on which no other agent had any effect. Methyl paraben alone was very effective against two organisms (Aspergillus and Bacillus), and slightly inhibitory on three others. The combination of methyl paraben & propyl paraben is as effective as one alone.

The two other antimicrobials (diacetyl and natamycin) are not as effective alone as the parabens. When they are in combination with one of the parabens, inhibition was equal to or less than the paraben by itself. Diacetyl or natamycin does not contribute any synergistic effect.

Results on the organisms are summarized as follows:

Aspergillus niger

0.2 % methyl paraben and 0.2% propyl paraben are most effective against growth, whereas both parabens at 0.1% each was only mildly effective.

Bacillus stearothermophilus

The control has lower growth levels at 24 hours than the test samples, but methyl paraben, and a combination of both parabens, showed the greatest amount of inhibition. Other agents are only slightly effective in slowing growth.

Candida albicans

0.2% propyl paraben shows good inhibition. The combination of 0.1% methyl paraben and 0.1% propyl paraben, and 0.2% methyl paraben has a slight inhibitory effect.

Enterobacter cloacae

The test composition with 0.2% methyl paraben slows growth slightly in the first 24 hours, but otherwise growth is uninhibited.

Lactobacillus leichmanni

0.2% methyl paraben and 0.2% propyl paraben are effective.

Staphylococus aureus

0.2% propyl paraben shows a slight inhibitory effect in the first 24 hours.

### EXAMPLE 3

Test demonstrating increased lifetime of feeding tube (in vitro) when enteral feeding solution has 0.1% propyl paraben vs. no paraben. Feeding solution is perfused through a feeding tube and maintained at approximately 30° C. during the test, which may last several days or weeks. At the time the test is begun the solution within the feeding tube is inoculated with 100 CFU of Candida albicans. After a few hours of incubation (no flow in the tube), perfusion is started and continued until the tube becomes clogged due to overgrowth of the microorganisms.

Graph 1 illustrates the results of this test wherein 0.1% propyl paraben containing formula extended the time when clogging occurred by 1 to 2 weeks beyond that attained with no paraben.

### EXAMPLE 4

The antimicrobials, propyl paraben, sodium benzoate, and potassium sorbate are challenged singly, and in combinations, against 5 organisms. Results are consistent within the same challenge organism trial, and inhibition using a combination of all three antimicrobials is also effective on the variety of organisms tested.

Isosource HN® is packaged and retorted with the allowable FDA limits. Three samples, A, B, or C are prepared, having, respectively, 0.13% propyl paraben (A), 0.1% sodium benzoate (B), and 0.1% potassium sorbate (C). Additional samples D, E, and F are mixtures of A & B, A & C, and A & B & C. Three control samples: G having 0.26% ethanol; H having no preservatives and 1, a blank control are also prepared. A 350-ml aliquot of each retorted product is transferred to a sterile flask and inoculated with one of the designated organisms. A like trial is initiated for each of five organisms, Aspergillus niger, Candida albicans, Enterobacter cloacae, Staphylococcus aureus, and Lactobacillus delbrueckii. These organisms are inoculated, respectively, at 44 organisms/ml product; 2 organisms/ml product; 2 organisms/ml product; 4.1 organisms/ml product, and 30 organisms/ml product. An ethanol control is included for all the tests because it is used as the solvent carrier for paraben. An inoculated control with no inhibitors is also used for comparison. The samples are incubated at the appropriate temperature for optimum growth, and aliquots are pulled for testing at the designated intervals. Results are read and recorded as the population per milliliter of sample tested.

Results of the Specific Organism Test Runs as follows:

1. Aspergillus niger growth is inhibited most by propyl paraben than the other antimicrobials. At 7 days, only propyl paraben samples do not exhibit spore growth, and at 10 days, spores are visible on all samples. When spores formed, the sample is noted as “overgrown” and no counts are done, since spore growth compromises exponential growth and skews the data. The ethanol promotes growth in this test, as the inoculated control with no inhibitors have a count 4+ log cycles lower than the inoculated ethanol control at three days.

2. Candida albicans is inhibited most by the combination of all 3 antimicrobials. The ethanol also stimulates the yeast, as evidenced by a count 3 log cycles greater than the inoculated blank control after 3 days. Potassium sorbate alone is not effective, but in conjunction with propyl paraben, results are lower than propyl paraben by itself.

3. Enterobacter cloacae is inhibited most by the combination of all 3 antimicrobials. Potassium sorbate is the most effective agent alone, and more effective with propyl paraben than with sodium benzoate. Two inoculated controls (ethanol and blank) coagulated before the end of the trial and are not tested further.

4. Staphylococcus aureus shows the greatest inhibition of the 3 bacteria with the combination of all three antimicrobials. There is only a 3 log cycle increase in the population over the 48 hour period. This organism also exhibits some inhibition from the ethanol control.

5. Lactobacillus delbrueckii exhibits inhibition at 24 hours very similar to the other bacteria, but at 48 hours, there is less than ½ log cycle difference between all the populations. The inoculated control (with no inhibitors) is in the death phase at 48 hours, as the population peaks between 24 and 48 hours, having a decreased count at 48 hours. All the other counts are still increasing at 48 hours, with the combinations of inhibitors showing a slightly better effect than any substance used alone.

Propyl paraben by itself is more effective against 4 of the organisms than either of the other 2 antimicrobials alone. The combination of any 2 inhibitors is more effective than any of them used singly. Overall, the combination of all 3 antimicrobials is the most effective in 4 of the five challenges demonstrating an inhibitory synergism.

What is claimed is:

1. A composition comprising an enteral complete feeding solution and from 0.05 to 1.0% by weight C₁₋₁₀-alkyl paraben in combination with 0.1-0.2% by weight of the pharmaceutically acceptable salts of benzoic and sorbic acids.

2. The composition of claim 1 having a pH is 3-8.

3. The composition of claim 1 having a pH is 2-6.9.

4. The composition of claim 1 having a pH>5.5.

5. The composition of claim 1 in which the alkyl paraben is methyl paraben

6. The composition of claim 1 in which the alkyl paraben is ethyl paraben.

7. The composition of claim 1 in which the alkyl paraben is propyl paraben.

8. The composition of claim 1 comprising 0.1-0.2% by weight alkyl paraben.

9. The composition of claim 1 comprising the sodium or potassium salts of benzoic and sorbic acids.

10. The composition of claim 1 further comprising a thickener in a concentration in the range of 0.50% to 12.0% by weight, wherein the thickener is xanthan gum or carrageenan.

11. An enteral feeding tube system comprising an enteral feeding tube and a composition of claim 1.

12. The system of claim 11 comprising methyl paraben and the sodium or potassium salts of benzoic and sorbic acids.

13. A method of inhibiting the growth of Aspergillus niger, Candida albicans, Enterobacter cloacae, Staphylococcus aureus, and Lactobacillus delbrueckii in an enteral complete feeding solution which comprises adding to said solution a composition which comprises 0.05 to 1.0% by weight C₁₋₁₀-alkyl paraben in combination with 0.1-0.2% by weight of the pharmaceutically acceptable salts of benzoic and sorbic acids.

14. A method of claim 13 wherein the alkyl paraben is methyl or propyl paraben.

15. A method of claim 13 wherein the combination has a pH of 3-8 and comprises the sodium or potassium salts of benzoic and sorbic acids.
